(19) United States
(12) Patent Application Publication
(10) Pub. No.: US 2023/0023276 A1
(43) Pub. Date: Jan. 26, 2023

(54) 3-D BIOPRINTING COMPRISING BIOLOGICALLY-RELEVANT MATERIALS AND RELATED METHODS

(71) Applicant: UNIVERSITY OF LOUISVILLE RESEARCH FOUNDATION, INC., Louisville, KY (US)

(72) Inventors: Stuart K. WILLIAMS, Louisville, KY (US); Brian C. GETTLER, Louisville, KY (US); Piyani S. GANDHI, Louisville, KY (US)

(21) Appl. No.: 17/862,892

(22) Filed: Jul. 12, 2022

Related U.S. Application Data
(63) Continuation-in-part of application No. 16/312,042, filed on Dec. 20, 2018, now Pat. No. 11,419,962.
(60) Provisional application No. 62/354,929, filed on Jun. 27, 2016.
(60) Provisional application No. 62/422,694, filed on Nov. 16, 2016.

(51) Int. Cl. C12N 5/00; C12N 5/0775; C12N 5/0735; A61L 27/52; A61L 27/54; A61L 27/22; A61L 27/38; A61L 27/50; A61L 27/18; B33Y 10/00; B33Y 70/00

(52) U.S. Cl. CPC C12N 5/0062; C12N 5/0667; C12N 5/0606; A61L 27/52; A61L 27/54; A61L 27/22; A61L 27/3834; A61L 27/3804; A61L 27/50; A61L 27/18; B33Y 10/00; B33Y 70/00; C12N 2513/00; C12N 2533/30; C12N 2539/00; A61L 2300/252; A61L 2300/62; A61L 2430/20

(57) ABSTRACT

The present disclosure provides a method of bioprinting a 3-D structure comprising one or more biologically-relevant materials on a super-hydrophobic surface. In one embodiment, the method comprises providing a composition having one or more biologically-relevant materials dispersed within a biocompatible medium. A pattern comprising a hydrophilic material is deposited on a defined area of the super-hydrophobic surface, wherein the pattern is modeled after a biological structure. The composition having the one or more biologically-relevant materials is then bioprinted atop the hydrophilic surface to form a 3-D structure, wherein the hydrophilic surface maintains the 3-D structure in a desired position or shape on the super-hydrophobic surface.

### CROSS-REFERENCE TO RELATED APPLICATION

This application claims priority from U.S. application Ser. No. 16/312, 042, which is the national stage application of PCT/US2017/039483, filed Jun. 27, 2017, which claims priority of U.S. Provisional Application Ser. No. 62/354,929, filed Jun. 27, 2016, and U.S. Provisional Application Ser. No. 62/422,694, filed Nov. 16, 2016, the entire disclosures of these applications are incorporated herein by this reference.

### FIELD OF THE INVENTION

The present disclosure relates in general to the field of 3-D bioprinting. In certain embodiments, the present disclosure provides compositions and methods of 3-D bioprinting structures of defined shape on superhydrophobic surfaces that contain hydrophilic lines or surfaces.

### BACKGROUND OF THE INVENTION

As it is discussed in a recent review article (Dey and Ozbolat, Sci. Rep. 2020), the first 3D printer was built in the early 1980s, capable of creating solid objects by following a computer-aided design (CAD). By the late 1990s, 3D printing made its appearance in healthcare where surgeons began 3D printing dental implants, custom prosthetics, and kidney bladders. Subsequently the term ‘3D bioprinting’ emerged where the material being printed, called ‘bioink’, consisted of living cells, biomaterials, or active biomolecules. 3D bioprinting involves layer-by-layer deposition of bioink to create 3D structures, such as tissues and organs. Apart from organ printing, bioprinting is also being used to fabricate in-vitro tissue models for drug screening, disease modelling, and several other in-vitro applications. A review on bioinks suitable for 3D bioprinting can be found in Williams and Hoying, Bioinks for Bioprinting, K. Turksen (ed.), Bioprinting in Regenerative Medicine, Stem Cell Biology and Regenerative Medicine, Springer International Publishing, 2015.

3D bioprinting can be broadly categorized as either extrusion, droplet, or laser-based bioprinting. Extrusion based bioprinting employs mechanical, pneumatic or solenoid dispenser systems to deposit bioinks in a continuous form of filaments, while droplet based bioprinting relies on the generation of bioink droplets by thermal, acoustic or electrical stimulation. The selection of “bioinks” for each of these different bioprinting modalities usually varies based on the ink's rheology, viscosity, crosslinking chemistry, and biocompatibility. Extrusion based bioprinting primarily requires shear thinning bioinks while droplet or inkjet bioprinting needs materials with low viscosity. Over the past few years, the design and synthesis of bioinks has evolved to meet the increasing needs of new bioprintable materials.

Even though 3D bioprinting is advancing at a commendable rate with researchers trying to develop new printing modalities as well as improve existing modalities, there still remains a multitude of challenges that need to be overcome. Currently, a limited number of bioinks exist which are both bioprintable and which accurately represent the tissue architecture needed to restore organ function post-printing. Moreover, the bioprinting process itself needs to be more cell friendly. Effective techniques need to be developed for high throughput generation and bioprinting of organoids for personalized drug testing and predictive disease models. Thus, there is a need in the art for improved 3D bioprinting methodologies.

### SUMMARY OF THE INVENTION

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this summary or not. To avoid excessive repetition, this summary does not list or suggest all possible combinations of such features.

In some implementations of the presently-disclosed subject matter, methods of making 3-D structures comprising biologically-relevant materials are provided. In one implementation, a method of making a 3-D structure including one or more biologically-relevant materials is provided in which a composition is first created or provided, where the composition includes one or more biologically-relevant materials dispersed within a biocompatible medium. An amount of a hydrophilic material is then deposited in a defined area and/or in a defined amount onto a super-hydrophobic surface of a suitable substrate. In some embodiments, the hydrophilic material is deposited in a pattern modeled after a biological structure. In some implementations, the hydrophilic material deposited on the super-hydrophobic surface is comprised of a polyoxyethylene-polyoxypropylene block copolymer. In some implementations, the super-hydrophobic surfaces utilized in accordance with the presently-disclosed subject matter have a water contact angle of greater than about 150°, such as, in some implementations, a water contact angle of about 150° to about 170°.

Regardless of the particular hydrophilic materials and/or water contact angles of the super-hydrophobic surfaces utilized in an exemplary method of the presently-disclosed subject matter, once the composition and substrate are produced, the composition is then bioprinted (e.g., by direct write printed) directly onto the hydrophilic material positioned on the super-hydrophobic surface to thereby produce a 3-D structure comprising the biologically-relevant materials. In some embodiments, subsequent to bioprinting the composition, the resulting 3-D structure can then be incubated at physiological temperatures for a period of time while maintaining the shape of the 3-D structure. In some implementations, if desired, the 3-D structure can then be further cultured in a cell culture medium.

In some implementations of the presently-disclosed methods, the one or more biologically-relevant materials included in an exemplary 3-D structure comprise magnetic beads, stromal vascular fraction cells, stem cells, one or more relevant cells, groups of cells or tissues, or combinations thereof. For example, in some implementations, a 3-D structure can be produced comprising stromal vascular fraction cells in combination with one or more relevant cells, such as pancreatic islet cells. In some implementations, the one or more biologically-relevant materials can thus comprise stromal vascular fraction cells. In some implementations, the one or more biologically-relevant materials comprise one or more islet cells.

With respect to the biocompatible medium used to form the suspensions utilized in the presently-disclosed methods, in some implementations, the biocompatible medium comprises a hydrogel. In some implementations, the hydrogel is comprised of a material selected from the group consisting of agarose, alginate, collagen, a polyoxyethylene-polyoxypropylene block copolymer; silicone, polysaccharide, polyethylene glycol, and polyurethane. In some implementations, the hydrogel is comprised of collagen type I.

These and other aspects of the invention will be appreciated from the ensuing descriptions of the figures and detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIG. 1 presents one embodiment of bioprinting hydrogel rods or tubes on a superhydrophobic surface using a hydrophilic surface (e.g. a thin rod of Pluronic) to maintain position after extrusion.

FIGS. 2A-2E show examples of different shapes of polyoxomer printed on a hydrophobic surface. FIGS. 2A-2C shows rods of Pluronic printed on a surface using pen tips of descending inner diameters. The size of the pen tip and the conditions of printing (e.g. pressure) regulates the quantity of Pluronic extruded. FIG. 2D shows interconnected lines. FIG. 2E shows a complex interconnected structure.

FIG. 3 presents one embodiment of taking a biologic image, converting the image to a CAD design, and manufacturing the image into a Pluronic F127 printed structure. Left panel: biological image of a left ventricular Purkinje system; Middle panel: CAD design of the Purkinje system; Right panel: Pluronic F127 printed structure on a hydrophobic surface.

FIGS. 4A-4D presents examples of hydrogel rods printed on hydrophilic surfaces. Representative images of collagen-alone lines printed from left to right by using a 25-gauge (FIGS. 4A and 4B) or 33-gauge (FIGS. 4C and 4D) pen tip at a linear speed of 10 mm/s (FIGS. 4A and 4C) or 20 mm/s (FIGS. 4B and 4D) with pressure settings ranging from 2 to 20 psi. The results show that without a superhydrophobic surface the material spreads onto the surface and does not maintain shape.

FIGS. 5A-5C show BAEC plus Col I constructs generated on the basis of anatomical structure. (FIG. 5A) Angiogram of a pig heart was used to develop a script to direct the BAT to extrude four layers of solution in the pattern of the LAD and its four diagonals (black lines). (FIG. 5B) Image of BAEC plus Col I construct coextruded according to the angiogram script, as seen on day 0. (FIG. 5C) Construct 2 h after extrusion.

### DETAILED DESCRIPTION OF THE INVENTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

In some implementations of the presently-disclosed subject matter, methods of making a 3-D structure are thus provided. In some implementations, a method of making a 3-D structure is provided in which a hydrophilic material is first placed onto a defined area on the surface of a substrate where the surface of the substrate is super-hydrophobic. In some embodiments, the hydrophilic material is deposited in a pattern modeled after a biological structure (e.g. the Purkinje system in the heart. Other examples include the macrocirculation of the heart (i.e. the cardiac coronary vascular system where the blood vessels are printed as tubes based on the coronary vessel architecture.

In some embodiments, the modeling after a biological structure comprises computer-aided design (CAD). One or more biologically-relevant materials are then suspended within a biocompatible medium to create a composition, which can be bio-printed onto the hydrophilic material. For instance, in one exemplary implementation of a method for making the presently-disclosed subject matter makes use of direct-write printing as a form of bioprinting, e.g. a BioArchitecture Tool (BAT; see, e.g., U.S. Pat. No. 7,857,756; see also Smith, et al., Tissue Eng. 2004; 10:1566-1576, both of which are incorporated herein by this reference). In some embodiments, use of a computer-controlled stage is utilized, which not only permits independent X- and Y-axis translation, but also permits Z-axis movement of one or more translational print head/dispensing systems. In this regard, bioprinting parameters can first be scripted as printing instructions and then uploaded to the printing tool such that the printing tool (i.e., the BAT) can be used to produce a precise structure. In some implementations, by making use of such a printing tool, the size of the structure printed by such a system can be controlled by controlling the size of the pen used to print the droplet and by controlling the pressure with which the droplet is extruded from the pen. In some embodiments, about a 15 gauge pen to about a 25gauge pen and a pressure of about 2 psi to about 7 psi can be used to produce a droplet. In some implementations, the droplets have a diameter of about 1 mm to about 5 mm, about 2 mm to about 4 mm, or about 3 mm to about 4 mm. In some implementations, the size of the droplets is controlled by adjusting one or more parameters selected from the group consisting of: the viscosity of the suspension, the size of the delivery pen tip, the pressure used to extrude the suspension from the delivery pen, and the amount of time pressure is applied to the suspension in the delivery pen. Such parameters can readily be adjusted by those skilled in the art to produce a droplet or spheroid having a desired size.

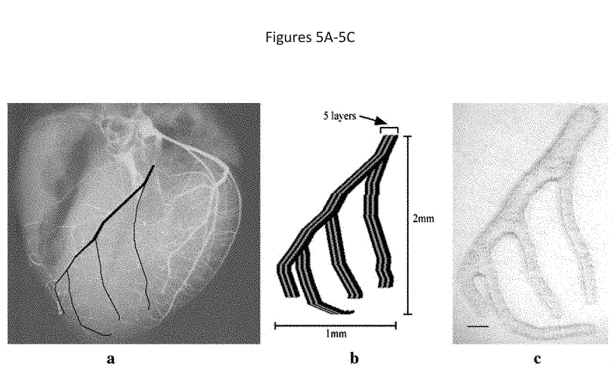

FIGS. 5A-C present one embodiment of how an array of the coronary system of the heart can be converted to a “script”—CAD design and then a collagen solution can be printed onto a surface using this design as a guide. Since the surface here is not a hydrophobic surface the collagen spreads on the surface.

As one exemplary implementation of a method for making a 3-D structure including one or more biologically-relevant materials, a 3-D structure is produced by first placing a suspension in the form of a cell suspension (e.g., a cell suspension comprised of a human stromal vascular fraction cell population mixed in collagen type I), in a delivery pen that is comprised of a hollow needle or tube-like structure. Extrusion of the biological suspension from the delivery pen is then controlled by increasing the pressure in the delivery pen to a specific value, thereby causing a droplet to form. The delivery pen is then lowered toward a hydrophilic material placed on a superhydrophobic surface of a substrate at a predetermined rate (e.g., 5 mm/sec). Upon contacting the hydrophilic material, the suspended droplet is subsequently attracted to the hydrophilic material and is released from the pen to thereby form the 3-D structure atop the hydrophilic spot on the super-hydrophobic surface. In some implementations, subsequent to bioprinting the suspension, the resulting 3-D structure can then be incubated at physiological temperatures (e.g., 37° C.) for a period of time, such as a period of time sufficient to polymerize the biological medium being utilized. In some implementations, if desired, the 3-D structure can then be further cultured in a cell culture medium.

The term “suspension” is used herein to refer to a composition comprising biologically-relevant materials, for example, magnetic particles, cells, tissues, proteins, and the like that are dispersed within a biocompatible medium. A suitable biocompatible medium for use in accordance with the presently-disclosed subject matter can typically be formed from any biocompatible material that is a gel, a semi-solid, or a liquid, such as a low-viscosity liquid, at room temperature (e.g., 25° C.) and can be used as a three-dimensional substrate for cells, tissues, proteins, and other biological materials of interest. Exemplary materials that can be used to form a biocompatible medium in accordance with the presently-disclosed subject matter include, but are not limited to, polymers and hydrogels comprising collagen, fibrin, chitosan, MATRIGEL™ (BD Biosciences, San Jose, Calif.), polyethylene glycol, dextrans including chemically-crosslinkable or photo-crosslinkable dextrans, and the like, as well as electrospun biological, synthetic, or biological-synthetic blends. In some implementations, the biocompatible medium is comprised of materials that support endothelialization, see, e.g., U.S. Pat. Nos. 5,744,515 and 7,220,276, both of which are incorporated herein by reference. In some implementations, the biocompatible medium is comprised of a hydrogel.

The term “hydrogel” is used herein to refer to two- or multi-component gels comprising a three-dimensional network of polymer chains, where water acts as the dispersion medium and fills the space between the polymer chains. Hydrogels used in accordance with the presently-disclosed subject matter are generally chosen for a particular application based on the intended use of the structure, taking into account the printing parameters that are to be used as well as the effect the selected hydrogel will have on the behavior and activity of the biological materials (e.g., cells) incorporated into the biological suspensions that are to be placed in the structure. Exemplary hydrogels of the presently-disclosed subject matter can be comprised of polymeric materials including, but not limited to: alginate, collagen (including collagen types I and VI), fibrinogen, elastin, keratin, fibronectin, proteoglycans, glycoproteins, polylactide, polyethylene glycol, polycaprolactone, polycolide, polydioxanone, polyacrylates, polyurethanes, polysulfones, peptide sequences, proteins and derivatives, oligopeptides, gelatin, elastin, fibrin, laminin, polymethacrylates, polyacetates, polyesters, polyamides, polycarbonates, polyanhydrides, polyamino acids carbohydrates, polysaccharides and modified polysaccharides, and derivatives and copolymers thereof as well as inorganic materials such as glass such as bioactive glass, ceramic, silica, alumina, calcite, hydroxyapatite, calcium phosphate, bone, and combinations of all of the foregoing. For additional information regarding the materials from which a hydrogel of the presently-disclosed subject matter may be comprised, see, e.g., U.S. Pat. Nos. 7,919,11, 6,991,652 and 6,969,480, each of which are incorporated herein by this reference.

With further regard to the hydrogels, in some implementations, the hydrogel is comprised of a material selected from the group consisting of agarose, alginate, collagen type I, a polyoxyethylene-polyoxypropylene block copolymer (e.g., Pluronic® F127 (BASF Corporation, Mount Olive, N.J.)), silicone, polysaccharide, polyethylene glycol, and polyurethane. In some implementations, the hydrogel is comprised of alginate. In some implementations, the hydrogel is comprised of collagen type I.

As used herein, the phrase “biologically-relevant materials” is used to describe materials that are capable of being included in a biocompatible medium as defined herein and subsequently interacting with and/or influencing biological systems. For example, in some implementations, the biologically-relevant materials are magnetic beads (i.e., beads that are magnetic themselves or that contain a material that responds to a magnetic field, such as iron particles) that can be combined with a hydrogel and then bioprinted along with the hydrogel to produce structure having a defined size that can be used in the calibration of instrumentation or for the separation and purification of cells and tissues according to methods known to those skilled in the art. As another example, in other implementations, the biologically-relevant materials include one or more cells and tissues, such that combining the cells or tissues with an appropriate biocompatible medium results in the formation of a cell or tissue suspension. In some implementations, the biologically-relevant materials are comprised of stromal vascular fraction cells, stem cells, one or more relevant cells, or combinations thereof. In some implementations, the biologically-relevant materials are comprised of stromal vascular fraction cells.

With respect to the stromal vascular fraction cells used in accordance with methods of the presently-disclosed subject matter, the stromal vascular fraction cells are those that are typically obtained by enzymatically digesting an amount of adipose tissue obtained from a subject, followed by a period of centrifugation to pellet the stromal vascular fraction of the adipose tissue. In this regard, the stromal vascular fraction contains a number of cell types, including endothelial cells, smooth muscle cells, pericytes, preadipocytes, mesenchymal stem cells (MSCs), endothelial progenitor cells, T cells, B cells, mast cells, and adipose tissue macrophages, as well as small blood vessels or microvascular fragments found within the stromal vascular fraction. For further explanation and guidance regarding the disassociation of adipose tissue to produce a stromal vascular fraction, see, e.g., U.S. Pat. No. 4,820,626, the entire contents of which are incorporated herein by this reference. In some embodiments, incomplete digestion of adipose tissue can also be used to yield adipose microvascular fragments, see, e.g., U.S. Pat. No. 7,029,838, which is also incorporated herein by reference.

With respect to the stem cells that can be utilized in accordance with the methods of the present invention, as used herein, the term “stem cells” refers broadly to traditional stem cells, progenitor cells, preprogenitor cells, precursor cells, reserve cells, and the like. Exemplary stem cells include, but are not limited to, embryonic stem cells, adult stem cells, pluripotent stem cells, neural stem cells, liver stem cells, muscle stem cells, muscle precursor stem cells, endothelial progenitor cells, bone marrow stem cells, chondrogenic stem cells, lymphoid stem cells, mesenchymal stem cells, hematopoietic stem cells, central nervous system stem cells, peripheral nervous system stem cells, and the like. Descriptions of stem cells, including methods for isolating and culturing them, may be found in, among other places, Embryonic Stem Cells, Methods and Protocols, Turksen, ed., Humana Press, 2002; Weisman et al., Annu. Rev. Cell. Dev. Biol. 17:387-403; Pittinger et al., Science, 284:143-47, 1999; Animal Cell Culture, Masters, ed., Oxford University Press, 2000; Jackson et al., PNAS 96(25):14482-86, 1999; Zuk et al., Tissue Engineering, 7:211-228, 2001; and U.S. Pat. Nos. 5,559,022, 5,672,346 and 5,827,735.Descriptions of stromal cells, including methods for isolating them, may be found in, among other places, Prockop, Science, 276:71-74, 1997; Theise et al., Hepatology, 31:235-40, 2000; Current Protocols in Cell Biology, Bonifacino et al., eds., John Wiley & Sons, 2000; and U.S. Pat. No. 4,963,489. One of ordinary skill in the art will understand that the stem cells and/or stromal cells that are selected for inclusion in a tissue construct are typically selected when such cells are appropriate for the intended use of a particular construct.

Finally, with respect to the relevant cells that can be utilized in accordance with the methods of the present invention, the term “relevant cells,” as used herein refers to cells that are appropriate for incorporation into a structure of the presently-disclosed subject matter, based on the intended use of that structure. In some embodiments, the term “relevant cells” can be used interchangeable with the term “regenerative cells” as the relevant cells described herein have the ability to form a functional tissue following implantation. For example, relevant cells that are appropriate for the repair, restructuring, or repopulation of particular damaged tissue or organ will typically include cells or groups of cells that are commonly found in that tissue or organ. In that regard, exemplary relevant cells that can be incorporated into the presently-disclosed subject matter include neurons, cardiomyocytes, myocytes, vascular and/or gastrointestinal smooth muscle cells, chondrocytes, pancreatic acinar cells, islets of Langerhans, islet beta cells, osteocytes, hepatocytes, Kupffer cells, fibroblasts, myoblasts, satellite cells, endothelial cells, adipocytes, preadipocytes, biliary epithelial cells, and the like. These types of cells may be isolated and used immediately or subjected to culture by conventional techniques known in the art. Exemplary techniques can be found in, among other places; Freshney, Culture of Animal Cells, A Manual of Basic Techniques, 4th ed., Wiley Liss, John Wiley & Sons, 2000; Basic Cell Culture: A Practical Approach, Davis, ed., Oxford University Press, 2002; Animal Cell Culture: A Practical Approach, Masters, ed., 2000; and U.S. Pat. Nos. 5,516,681 and 5,559,022. In some implementations, the biologically-relevant cells comprise pancreatic islet cells (e.g. beta cells) or the entire intact islet.

Regardless of the particular type of biologically-relevant materials that are combined with a biocompatible medium in accordance with the presently-disclosed subject matter, as indicated above, once the biologically-relevant materials are combined with a biocompatible medium, a droplet of the resulting suspension is then bioprinted onto a hydrophilic material placed on a superhydrophobic surface. In this regard, when the suspension reaches room temperature, the suspension will typically gelate and form a structure having a more stable geometry. To maintain the geometry of the droplets after extrusion but before polymerization or gelation, however, and as noted above, the presently-disclosed methods make use of a substrate having a super-hydrophobic surface.

The term “super-hydrophobic” is used herein to refer to substrates exhibiting a minimal attraction to water. Super-hydrophobic surfaces typically exhibit the lotus effect such as what occurs when water droplets come into contact with, for example, lotus or taro leaves. Other naturally-occurring examples of super-hydrophobic surfaces supporting the formation of water droplets can be found in, for example, the fogstand beetle (Stenocara gracilipes), which is found in the Namib Desert. In this regard, such super-hydrophobic substrates or surfaces will typically have a water contact angle, or the angle where a liquid or vapor interface meets a solid surface as measured through the liquid, of greater than about 150°. In some implementations, the super-hydrophobic surfaces used herein have water contact angles of greater than 150°. In some implementations, the water contact angle of an exemplary super-hydrophobic surface is about 150° to about 170°. Numerous super-hydrophobic surfaces having such water contact angles are known to those skilled in the art and can be present as a result of the particular substrate utilized or as a result of a coating applied to the substrates. For example, in some implementations, a super-hydrophobic surface can be produced by spraying a water repellant coating, such as NEVERWET™ (Rust Oleum, Vernon Hills, Ill.), onto a suitable substrate. Further examples, of super-hydrophobic surface coatings include, but are not limited to, silica, manganese oxide polystyrene (MnO₂/PS), zinc oxide polystyrene (ZnO/PS), precipitated calcium carbonate, perfluorobutanesulfonic acid, carbon nanotube structures, paraffin, polytetrafluoroethylene, wax, and the like.

As also noted above, in some implementations of the methods described herein, an amount of a hydrophilic material, i.e., a material having an increased affinity for water and typically having a water contact angle of less than about 90°, is placed onto a defined area of the hydrophobic surface. The amount of hydrophilic material and the area onto which the hydrophilic material is placed can, of course, vary depending on the structure being produced. In some implementations, about 2 μl to about 5 μl of a hydrophilic material is placed onto a hydrophobic surface to ensure that the spheroid attaches to the super-hydrophobic surface rather than remaining attached to printing pen. In some implementations, block copolymers, such as Pluronic® F127, having an amphiphilic block structure can be utilized as such copolymers are both hydrophilic and hydrophobic and are thus capable of adhering to both the hydrophobic surface and aqueous biocompatible media, such as collagen. Other hydrophilic materials capable of use in accordance with the present invention include, but are not limited to, other copolymers such as P 188, as well as other materials such are urethanes and silanes. In some embodiments, hydrophilic materials that are useful in the formation of 3-D structure provide adhesion characteristics that are reversible to allow removal of the 3-D structure. Such a reversal can be caused by, among other things, a change in temperature or solubilization of the hydrophilic substance in the aqueous phase of the 3-D structure.

Still further provided, in some embodiments of the presently-disclosed subject matter, are 3-D structures made according to the methods described herein. Without wishing to be bound by any particular theory or mechanism, it is believed that such 3-D structures are advantageous as, for example: an in vitro assay of angiogenesis and vasculogenesis to screen drugs; a device that can be implanted into a patient to provide new blood flow to ischemic tissue; and a device that can be constructed using the adipose derived stem and regenerative cells and incorporates other parenchymal cells that can includes liver cells, muscle cells, fat cells, pancreatic cells including islets, brain cells, reproductive cells, kidney cells, and the like. Furthermore, it is believed that the presently-disclosed 3-D structures and methods allow for the production of a device that can be formed and implanted immediately without the need to subject material to tissue culture and without the need to utilize other additives (e.g. alginate) to support formation of a stable structure.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples. The following examples may include compilations of data that are representative of data gathered at various times during the course of development and experimentation related to the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

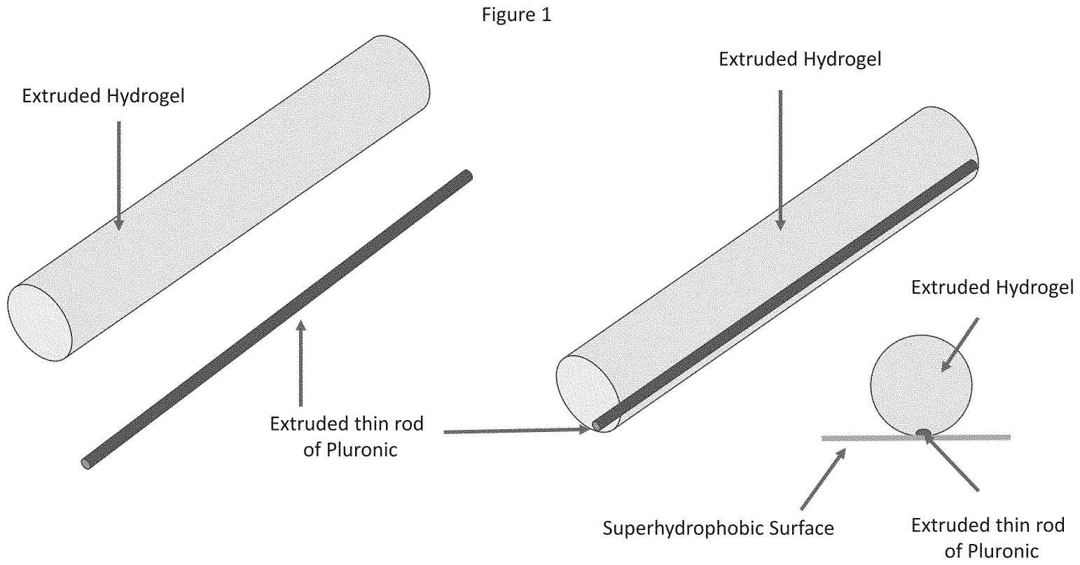

FIG. 1 presents one embodiment of bioprinting hydrogel rods or tubes on a superhydrophobic surface using a hydrophilic surface (e.g. a thin rod of Pluronic that is allowed to dry on the hydrophobic surface) to maintain the position and shape (e.g >150 degree contact angle of the rod/tube in cross section of the hydrogel after extrusion. Examples of hydrogels that can be used include, but are not limited to, collagen, fibrin, aqueous solutions (including water, saline), alginates.

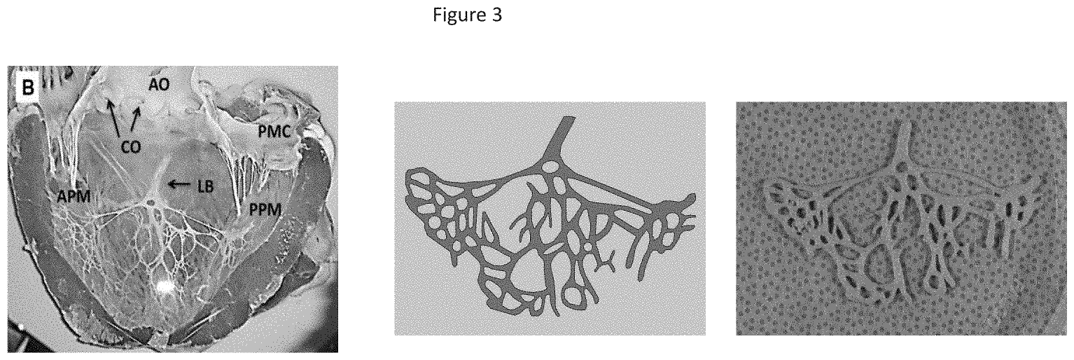

FIG. 3 presents one embodiment of taking a biologic image, converting the image to a CAD design, and manufacturing the image into a Pluronic F127 printed structure. In this example, the left ventricular Purkinje system is used as an example. Left panel: biological image of a left ventricular Purkinje system; Middle panel: a CAD design of the Purkinje system based on the image of the left panel; Right panel: a Pluronic F127 structure printed on a hydrophobic surface based on the CAD design of the middle panel. One of ordinary skill in the art would readily follow the same approach to bioprint other biological systems, such as the macro and microcirculations of the heart, kidney, liver, lung; the airway system of the lung, e.g trachea to the alveolar system, ligaments and tendons of the orthopedic system and the soft tissue implants used in plastic and reconstructive surgery. Another biologic system is tissue implants used in correcting the shape of the cornea and lens of the eye. In addition to CAD, other techniques well-known in the art can be used, for example, computer numerical control or computer assisted manufacturing.

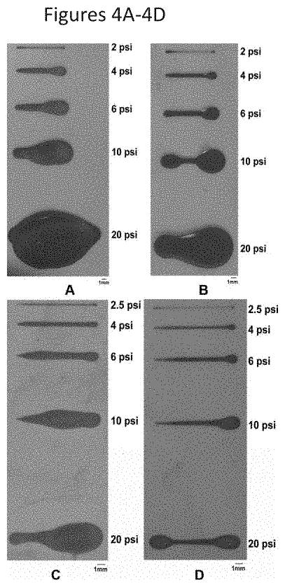

FIGS. 4A-4D presents examples of hydrogel rods printed on hydrophilic surfaces. This example demonstrates that when the hydrogels are printed directly onto a hydrophilic surface, the hydrogels do not maintain their shape. Hence, one objective of the present disclosure is to provide a method of bioprinting a 3-D structure comprising one or more biologically-relevant materials on a super-hydrophobic surface. In one embodiment, the method comprises providing a composition having one or more biologically-relevant materials dispersed within a biocompatible medium. A pattern comprising a hydrophilic material is deposited on a defined area of the super-hydrophobic surface, wherein the pattern is modeled after a biological structure. The composition having the one or more biologically-relevant materials is then bioprinted atop the hydrophilic surface to form a 3-D structure, wherein the hydrophilic surface maintains the 3-D structure in a desired position or shape on the super-hydrophobic surface.

In one embodiment, there is provided a method of making a 3-D structure comprising one or more biologically-relevant materials, comprising the steps of:

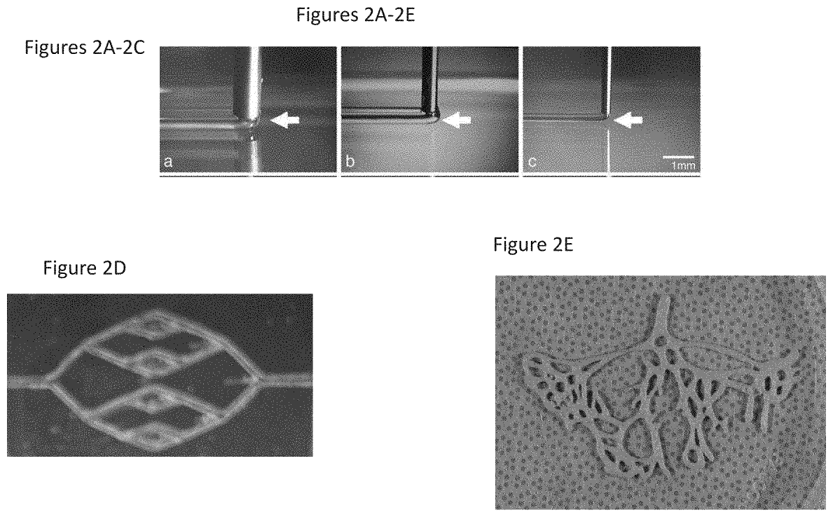

(i) depositing a pattern comprising triblock copolymer onto a super-hydrophobic surface to form a hydrophilic surface on the super-hydrophobic surface, wherein said pattern is modeled after a biological structure, and the triblock copolymer has an amphiphilic block structure which gives it hydrophilic and hydrophobic properties. In another embodiment, the pattern can also be modelled after non biologic structures such as linear bifurcated structures (FIG. 2D) or chaotic structures;

(ii) providing a composition comprising one or more biologically-relevant materials dispersed within a biocompatible medium; and

(iii) bioprinting said composition atop the hydrophilic surface to form a 3-D structure comprising said one or more biologically-relevant materials, wherein said hydrophilic surface maintains said 3-D structure in a desired position or shape on said super-hydrophobic surface. In one embodiment, the superhydrophobic surface constrains the printed rod/tube in a structure that maintains the contact angle consistently greater that 150 degrees.

In one embodiment, the biocompatible medium is a hydrogel. In one embodiment, the hydrogel comprises collagen type I.

In one embodiment, the above modeling after a biological structure comprises computer-aided design (CAD), CAM, or CNS.

In one embodiment, the biologically-relevant materials comprise stromal vascular fraction, microvascular fragments or stem cells. In one embodiment, the stem cells are embryonic stem cells, adult stem cells, or pluripotent stem cells. In another embodiment, the biologically-relevant materials comprise one or more cells appropriate for repair, restructure or repopulation of a tissue or organ. Examples of cells appropriate for repair, restructure or repopulation of a tissue or organ include, but are not limited to, neurons, cardiomyocytes, myocytes, vascular or gastrointestinal smooth muscle cells, chondrocytes, pancreatic acinar cells, islets of Langerhans, islet beta cells, osteocytes, hepatocytes, Kupffer cells, fibroblasts, myoblasts, satellite cells, endothelial cells, adipocytes, preadipocytes, or biliary epithelial cells.

In one embodiment, the above method further comprises the step of incubating the 3-D structure at physiological temperatures for a suitable period of time subsequent to bioprinting the 3-D structure. In another embodiment, the above method further comprises the step of culturing the 3-D structure in a cell culture medium subsequent to bioprinting the 3-D structure.

In another embodiment, another example of a rod structure was described in U.S. Pat. No. 10,889,799 (see FIG. 12 therein). While this example shows the printing of spheroids with an inner core of a cell product and an outer core of microvascular fragments, the same delivery pen can be used to print a rod containing the same materials. Tubes can also be printed on the hydrophilic/hydrophobic surface. In one embodiment, the bioprinting of the spheroid/rod can be performed in a manner that allows for the production of a pre-vascularized hydrogel spheroid. For instance, in some implementations, a method of making a pre-vascularized hydrogel spheroid is provided that includes the steps of providing a first suspension that includes one or more relevant cells dispersed within a biocompatible medium, and providing a second suspension that includes one or more microvascular fragments dispersed within a biocompatible medium. A bioprinter (e.g., the B.A.T. assembly described herein above) having a first delivery pen surrounded by a second delivery pen is then provided, and the first suspension is placed in the first delivery pen, while the second suspension is placed in the second delivery pen. The first suspension and the second suspension are then extruded from the first delivery pen and the second delivery pen, respectively, in a substantially simultaneous manner, such that a droplet is formed with the second suspension encapsulating the first suspension. In other words, by coextruding the first suspension and the second suspension from the first and second delivery pens at substantially the same time, a droplet is formed wherein a biocompatible medium containing one or more microvascular fragments surrounds a core that is comprised of a biocompatible medium containing one or more stromal vascular fraction cells, stem cells, and/or one or more relevant cells. In some embodiments, upon formation of the droplet, the droplet is then placed against a surface of a salt solution to form a pre-vascularized spheroid.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### EXAMPLES

### Example 1

Using a 3D Bioprinter to Form Hydrophilic Areas on Superhydrophobic Surfaces

Fabrication of a Super-hydrophobic Surface. In one embodiment, the super-hydrophobic surface was formed on a polystyrene 48 multi-well plates (Corning, Corning, N.Y.) and 35 mm petri dishes using a 2-step aerosol application of NEVERWET™ (Rust Oleum, Vernon Hills, Ill.). The first step was the application of a binder to the surface as a base coat, which air dried at room temperature for at least one hour. This was followed by the application of a top sheet composed of polydimethylsiloxane modified with hexamethyldisilazane to form the super-hydrophobic layer. The super-hydrophobic layer thickness was measured to be 0.07 mm. The top sheet was subsequently air-dried at room temperature for an additional hour. NEVERWET™ had a reported contact angle of 165° and a surface was considered super-hydrophobic beyond contact angles of 150°. The contact angle of both water and unpolymerized collagen in solution was measured via a side view photograph and subsequent contact angle measurement in ImageJ.

Creation of Hydrophilic Areas. In one embodiment, hydrophilic areas on the super-hydrophobic surface were created using a 3D bioprinter (Bio-Assembly Tool (BAT) 3-D printer; nScrypt, Inc., Orlando, Fla.) to extrude Pluronic F-127 (Sigma, St. Louis, Mo.). In one embodiment, for each hydrophilic spot or line, the BAT extruded a target volume of 2.5 μL (for a spot) and 10 μL/cm (for a line) of 3.8% (wt/wt) Pluronic F-127 in 1× phosphate buffered saline (PBS). With the BAT time-pressure extrusion system, this required 2.5 PSI with an exposure time of 100 ms (for a spot) and continuous (for a line) through a 25 G needle to create the appropriate extrusion force to dispense the target volume. These spots were then allowed to air dry for 30 minutes before use.

What is claimed is:

1. A method of making a 3-D structure comprising one or more biologically-relevant materials, comprising the steps of:
depositing a pattern comprising triblock copolymer onto a super-hydrophobic surface to form a hydrophilic surface on the super-hydrophobic surface, wherein said pattern is modeled after a biological structure, and the triblock copolymer has an amphiphilic block structure which gives it hydrophilic and hydrophobic properties;
providing a composition comprising one or more biologically-relevant materials dispersed within a biocompatible medium; and
bioprinting said composition atop the hydrophilic surface to form a 3-D structure comprising said one or more biologically-relevant materials, wherein said hydrophilic surface maintains said 3-D structure in a desired position or shape on said super-hydrophobic surface.

2. The method of claim 1, wherein the biocompatible medium is a hydrogel.

3. The method of claim 2, wherein the hydrogel comprises collagen type I.

4. The method of claim 1, wherein said modeling after a biological structure comprises computer-aided design (CAD).

5. The method of claim 1, wherein said biologically-relevant materials comprise stromal vascular fraction, microvascular fragments or stem cells.

6. The method of claim 5, wherein said stem cells are embryonic stem cells, adult stem cells, or pluripotent stem cells.

7. The method of claim 1, wherein said biologically-relevant materials comprise one or more cells appropriate for repair, restructure or repopulation of a tissue or organ.

8. The method of claim 7, wherein said one or more cells appropriate for repair, restructure or repopulation of a tissue or organ comprise neurons, cardiomyocytes, myocytes, vascular or gastrointestinal smooth muscle cells, chondrocytes, pancreatic acinar cells, islets of Langerhans, islet beta cells, osteocytes, hepatocytes, Kupffer cells, fibroblasts, myoblasts, satellite cells, endothelial cells, adipocytes, preadipocytes, or biliary epithelial cells.

9. The method of claim 1, further comprising the step of incubating the 3-D structure at physiological temperatures for a suitable period of time subsequent to bioprinting the 3-D structure.

10. The method of claim 1, further comprising the step of culturing the 3-D structure in a cell culture medium subsequent to bioprinting the 3-D structure.
